(19) United States
(12) Patent Application Publication
(10) Pub. No.: US 2010/0192953 A1
(43) Pub. Date: Aug. 5, 2010

(54) HYPERINFLATION SYSTEM

(75) Inventors: Gary C.J. Lee, Wu-Jia (TW); Ming-Ze Chen, I-Lan City (TW)

Correspondence Address: MERCHANT & GOULD PC, P.O. BOX 2903, MINNEAPOLIS, MN, 55402-0903, US

(73) Assignee: Galemed Corporation, Wu-Jia (TW)

(21) Appl. No.: 12/562,571

(22) Filed: Sep. 18, 2009

(30) Foreign Application Priority Data
Feb. 4, 2009 (TW) 098201667

(51) Int. Cl. A61M 16/08

(52) U.S. Cl. 128/205.13

(57) ABSTRACT

A hyperinflation system includes an airbag having an air inlet and an air outlet, and a one-piece multi-way connector including a base, a first tube section projecting from a first side of the base and connected to the air outlet, a second tube section projecting from a second side of the base oppositely of the first tube section, and a pressure gauge projecting from the second side of the base. A multi-port adaptor includes a first port connected fluidly to the second tube section, and a second port adapted for connection with a patient interface.

### CROSS-REFERENCE TO RELATED APPLICATION

This application claims priority of Taiwanese Application No. 098201667, filed on Feb. 4, 2009.

### BACKGROUND OF THE INVENTION

1. Field of the Invention

This invention relates to a hyperinflation system, more particularly to a hyperinflation system that can supply oxygen continuously and that can simultaneously facilitate viewing of an air pressure value.

2. Description of the Related Art

Referring to FIG. 1, a conventional hyperinflation system 1 is shown to include a squeezable airbag 11 having an air outlet 111, a bent tube 12 connected fluidly to the air outlet 111, a facial mask 13 connected fluidly to the bent tube 12, and a pressure gauge 14 provided on the bent tube 12 opposite to the facial mask 13. An air supply tube 15 has one end connected fluidly to the bent tube 12, and the other end connected to an oxygen supply unit (not shown). Air supplied by the oxygen supply unit is administered to the patient via the air supply tube 15, the bent tube 12, and the facial mask 13. Excess air inside the bent tube 12 is stored in the airbag 11. The airbag 11 may be suitably squeezed so that air inside the airbag 11 may flow through the bent tube 12 and the facial mask 13 to be forcibly breathed by the patient. The pressure gauge 14 indicates the level of air pressure flowing within the bent tube 12 to facilitate proper adjustment of the amount of airflow within the bent tube 12. However, since the pressure gauge 14 is large and is disposed opposite to the facial mask 13, when the patient is a child, the pressure gauge 14 covers a large portion of the patient's face, so that it is difficult for a medical professional to ascertain the patient' s condition when using the conventional hyperinflation system 1.

Referring to FIG. 2, another conventional hyperinflation system 2 is shown to include a squeezable airbag 21, a bent tube 22, a connecting tube 23 disposed between and interconnecting fluidly the airbag 21 and the bent tube 22, a tubular insert 231 adjacent to the connecting tube 23, a blocking plate 232 provided partially around the tubular insert 231, a cover body 233 covering openably the tubular insert 231, and a pressure gauge 24 inserted into the tubular insert 231. The blocking plate 232 aids in maintaining the pressure gauge 24 in an upright position. Further, an air supply tube 25 has one end extending through the airbag 21 and the connecting tube 23 into the bent tube 22, and the other end connected to an oxygen supply unit (not shown). Air supplied by the oxygen supply unit is administered to the patient via the air supply tube 25 and the bent tube 22, or is stored in the airbag 21. The airbag 21 is squeezed to force air inside thereof to flow through the bent tube 22 and the facial mask 16 so as to be forcibly breathed by the patient. However, since the pressure gauge 24 has to be separately installed, assembly and use of the conventional hyperinflation system 2 is troublesome. Further, since the pressure gauge 24 is long and is disposed opposite to the facial mask 16, the bent tube 22 cannot undergo an angular rotation of 360° as desired, so that use of the conventional hyperinflation system 2 is inconvenient.

### SUMMARY OF THE INVENTION

Therefore, the object of the present invention is to provide a hyperinflation system that is capable of overcoming the aforementioned drawbacks of the prior art.

According to this invention, a hyperinflation system comprises an airbag having an air inlet and an air outlet, a one-piece multi-way connector, and a multi-port adaptor. The one-piece multi-way connector includes a base, a first tube section projecting from a first side of the base and connected to the air outlet, a second tube section projecting from a second side of the base oppositely of the first tube section, and a pressure gauge projecting from the second side of the base. The multi-port adaptor includes a first port connected fluidly to the second tube section, and a second port adapted for connection with a patient interface.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a perspective view of a conventional hyperinflation system;

FIG. 2 is a perspective view of another conventional hyperinflation system;

FIG. 3 is an exploded perspective view of a hyperinflation system according to the preferred embodiment of the present invention;

FIG. 4 is a fragmentary sectional view of the preferred embodiment in an assembled state;

FIG. 5 is a view similar to FIG. 4, but illustrating a spring member in a compressed state;

FIG. 6 illustrates an arc-shaped vent hole in a position away from an arc-shaped aperture;

FIG. 7 illustrates the arc-shaped vent hole in a position slightly communicating with the arc-shaped aperture;

FIG. 8 illustrates the arc-shaped vent hole in a position communicating partially with the arc-shaped aperture;

FIG. 9 illustrates the arc-shaped vent hole in a position communicating fully with the arc-shaped aperture; and

FIG. 10 is a view similar to FIG. 5, but illustrating a cover body removed from a third port of a multi-port adaptor.

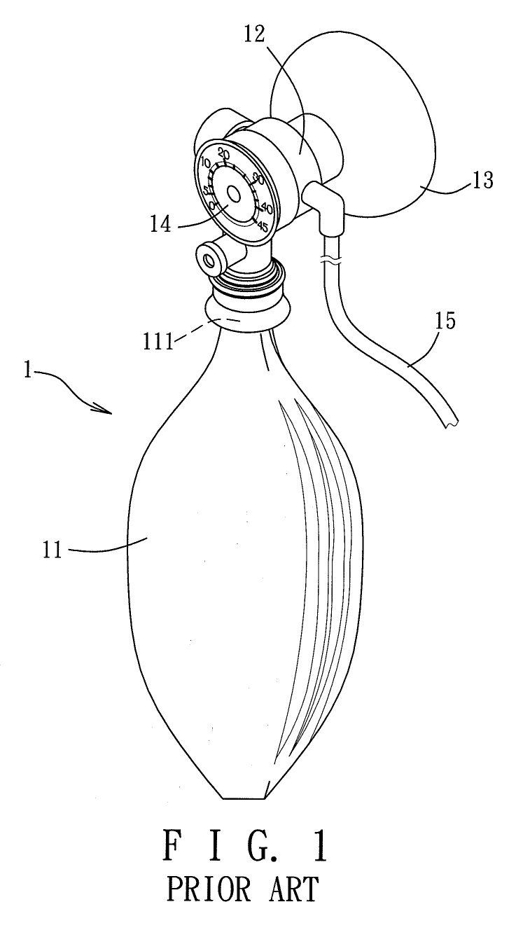

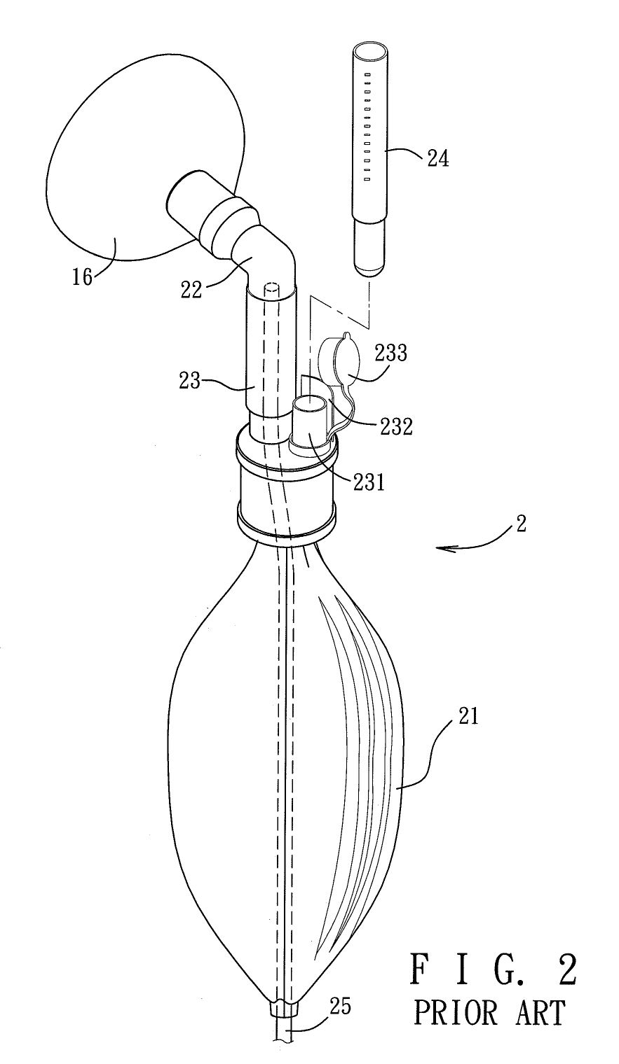

### DETAILED DESCRIPTION OF THE PREFERRED EMBODIMENT

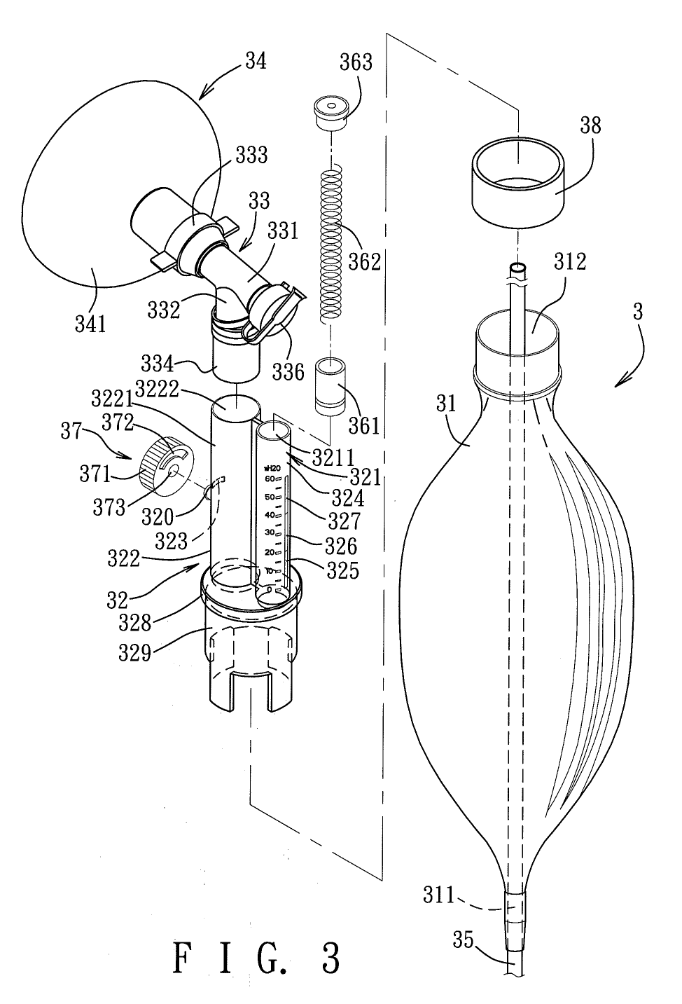

Referring to FIGS. 3 to 10, a hyperinflation system 3 according to the preferred embodiment of the present invention is shown to comprise a squeezable airbag 31, a one-piece multi-way connector 32, and a multi-port adaptor 33.

The squeezable airbag 31 has an air inlet 311 and an air outlet 312 opposite to each other.

The one-piece multi-way connector 32 includes a base 328, a transparent first tube section 329, a pressure gauge 321, and a transparent second tube section 322. The transparent first tube section 329 projects downwardly from a first side of the base 328, and is connected to the air outlet 312 so as to communicate fluidly with the airbag 31. The pressure gauge 321 projects upwardly from a second side of the base 328 oppositely of the first tube section 329, and has a transparent peripheral wall 324 defining a receiving space 3211 with a bottom opening communicating fluidly with the first tube section 329. An outer face of the peripheral wall 324 is provided with a graduated pressure scale and a column of pressure-indicating color levels 325, 326, 327 proximate to the graduated pressure scale. In this embodiment, the pressure-indicating color levels 325, 326, 327 are respectively green, yellow, and red in color for indicating respectively an inadequate amount of pressure, an adequate amount of pressure, and an excess amount of pressure. This facilitates a medical professional to quickly ascertain the amount of pressure that is being supplied to a patient by looking at the pressure-indicating color levels 325, 326, 327. The pressure gauge 321 includes an indicator 361 disposed movably in the receiving space 3211 and closing the bottom opening of the receiving space 3211, a spring member 362 disposed in the receiving space 3211 and having one end abutting against the indicator 361, and a plug member 363 disposed on top of the peripheral wall 324, covering a top opening of the receiving space 3211, and abutting against the other opposite end of the spring member 362.

The transparent second tube section 322 projects upwardly from the second side of the base 328 adjacent to the pressure gauge 321, and has a peripheral wall 3221 defining an air passage 3222 communicating fluidly with the first tube section 329, an arc-shaped aperture 323 formed in the peripheral wall 3221 and communicating fluidly with the air passage 3222, and a pivot pin 320 projecting outwardly from an outer face of the peripheral wall 3221 in proximity to the arc-shaped aperture 323.

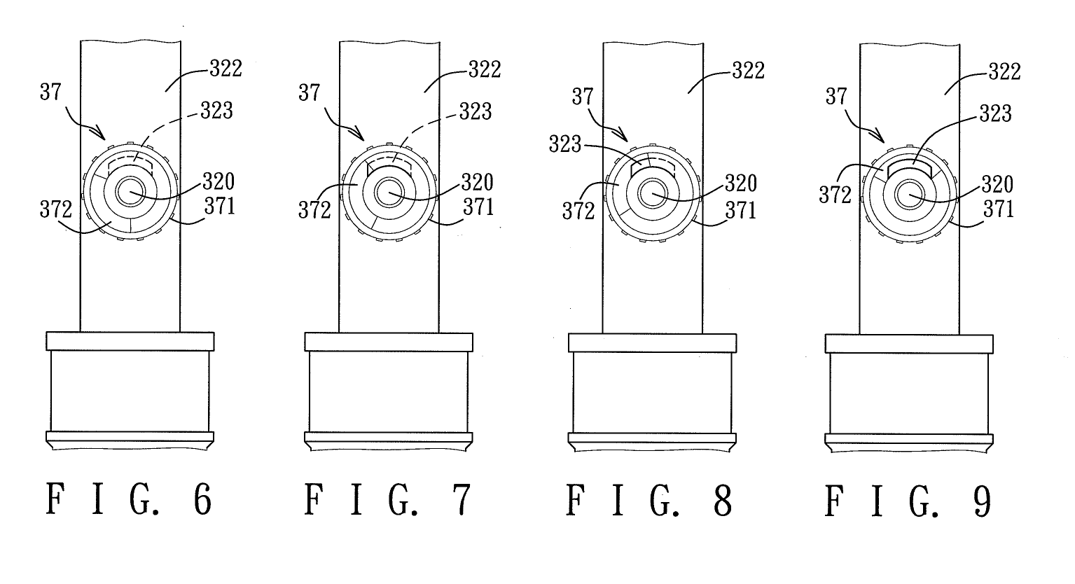

A pressure-releasing element 37 includes a knob 371 connected rotatably to the second tube section 322, an arc-shaped vent hole 372 formed in the knob 371 and alignable with the arc-shaped aperture 323, and a pivot hole 373 formed in the knob 371 in proximity to the arc-shaped vent hole 372. The pivot pin 320 is connected pivotally to the pivot hole 373 such that the knob 371 is rotatable relative to the second tube section 322 so as to adjust the position of the arc-shaped vent hole 372 relative to the arc-shaped aperture 323 to thereby control the amount of air to be released from the hyperinflation system 3. With reference to FIG. 6, the vent hole 372 may be disposed away from the aperture 323 so that no communication takes place between the same. With reference to FIG. 7, the knob 371 may be rotated to move the vent hole 372 to communicate slightly with the aperture 323, thereby permitting a small amount of air to be released from the system 3. With reference to FIG. 8, the knob 371 may be rotated further so that the vent hole 372 communicates partially with the aperture 323, thereby permitting a larger amount of air to be released from the system 3. With reference to FIG. 9, the vent hole 372 may be aligned to communicate fully with the aperture 323, thereby permitting an even larger amount of air to be released from the system 3.

The multi-port adaptor 33 has a T-tube body including a fourth tube section 331 having two opposite ends, a fifth tube section 332, and first and second connecting tubes 334, 333. The first connecting tube 334 is inserted into the second tube section 322. The fifth tube section 332 projects downwardly from the fourth tube section 331 between the two opposite ends thereof, and has an extension 3322 connected rotatably to the first connecting tube 334 and provided with an annular barbed end 3323 engaged to an inner surface of the first connecting tube 334, so that the fifth tube section 332 is rotatable by an angle of 360° relative to the second tube section 322. The extension 3322 is further provided with a first port 3321 that communicates fluidly with the air passage 3222.

The two opposite ends of the fourth tube section 331 are formed respectively with second and third ports 3311, 3312. The third port 3312 opens in a direction opposite to the second port 3311, is adapted for insertion of a suction tube (not shown), and is provided with a cover body 336 to openably close the third port 3312.

The second connecting tube 333 has one end connected rotatably to the fourth tube section 331 in proximity to the second port 3311 and rotatable by an angle of 360° relative to the fourth tube section 331, and the other end adapted for connection with a patient interface. The patient interface may be a facial mask, an endotracheal tube, or the like. In this embodiment, the patient interface is exemplified as a facial mask 34 having a mask body 341 covering the nose and mouth of the patient, and a tubular portion 342 connected fluidly to the second connecting tube 333.

The hyperinflation system 3 further comprises an air supply tube 35 having one end extending through the airbag 31 via the air inlet 311 and the air outlet 312, the first and second tube sections 329, 322, and into the first port 3321. The other end of the air supply tube 35 is adapted to be connected to an air supply source (not shown).

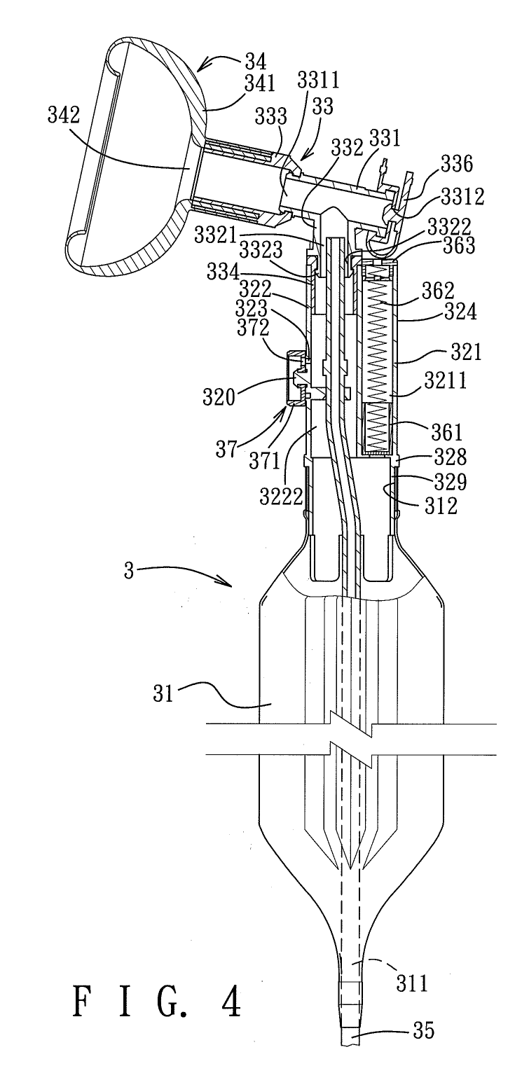

With reference to FIGS. 3 and 4, during assembly of the airbag 31, the multi-way connector 32, and the multi-port adaptor 33, the first tube section 329 is first inserted into the air outlet 312, and is secured thereto through the characteristics of the material of the airbag 31. A piece of adhesive tape 38 may be further used to strengthen the connection between the first tube section 329 and the airbag 31. The first connecting tube 334 is then press-fitted into a top end of the second tube section 322.

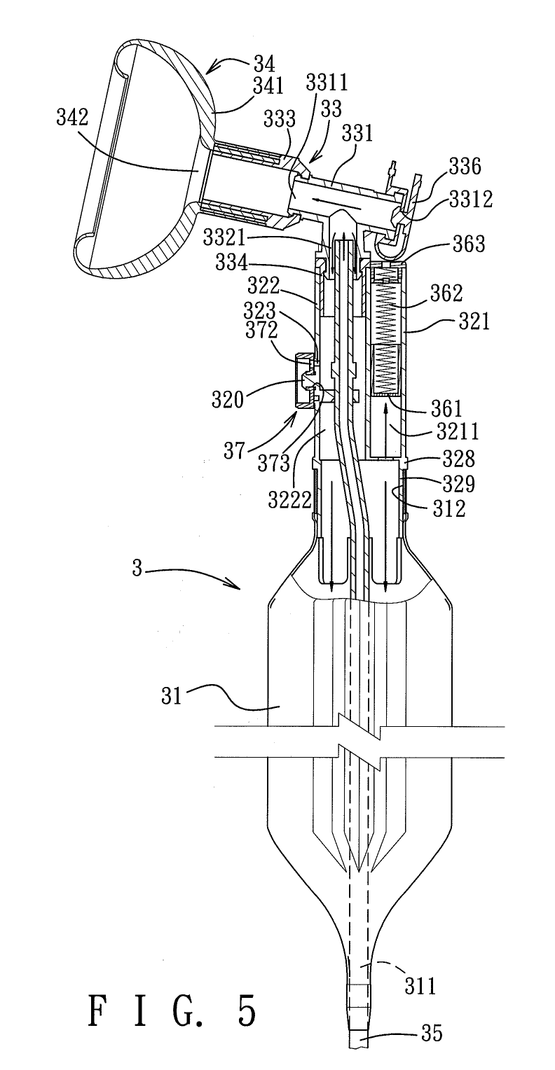

With reference to FIGS. 4 and 5, the hyperinflation system 3 of the present invention makes use of the air supply source to supply continuous air, which is usually oxygen, to the patient. The supplied air passes through the air supply tube 35 directly to the second connecting tube 333, and then to the facial mask 34 for inhalation of the patient. The excess air, which is not inhaled by the patient, flows back into the second tube section 322 through the first port 3321 and the first connecting tube 334, and is stored in the airbag 31. At this time, the medical professional can view the position of the indicator 361 that coincides with the graduated pressure scale on the outer face of the peripheral wall 324 of the pressure gauge 321 to ascertain the level of pressure inside the system 3. When the pressure is excessively high or excessively low, the knob 371 of the pressure-releasing element 37 is rotated selectively, as shown in FIGS. 6 to 9, so as to adjust the position of the arc-shaped vent hole 372 relative to the arc-shaped aperture 323. Hence, when necessary, a suitable amount of pressure can be released from the system 3. The hyperinflation system 3 can therefore be maintained at a suitable level of pressure, thereby enabling the patient to breathe smoothly and comfortably. Further, if necessary, the medical professional can squeeze the airbag 31 at the same time so that air in the airbag 31 can flow through the first and second tube sections 329, 322, the first and second ports 3321, 3311, and the facial mask 34 so as to be inhaled by the patient.

Moreover, because the fifth tube section 332 is rotatable by an angle of 360° relative to the second tube section 322, the medical professional, whether he/she be left-handed or right handed, can adjust the multi-way connector 32 relative to the fifth tube section 332 so as to obtain an optimal view of the pressure gauge 321. Thus, the optimal view of the pressure gauge 321 can be obtained according to one' s requirement by rotating the multi-way connector 32 relative to the multi-port adaptor 33.

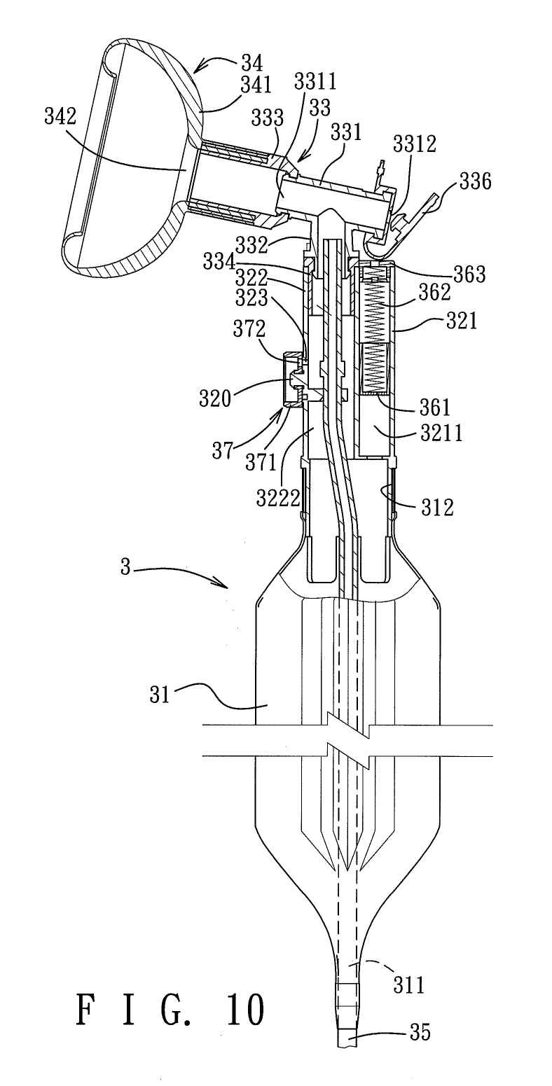

With reference to FIG. 10, when suctioning of the patient' s phlegm is desired, without influencing the continuous supply of air into the hyperinflation system 3, the medical professional simply opens the third port 3312 by removing the cover body 336 from the third port 3312, and the suction tube (not shown) can be inserted into the patient' s body through the third port 3312, the second port 3311, the second connecting tube 333, and the facial mask 34. Hence, use of the hyperinflation system 3 of the present invention is very convenient.

From the aforementioned description, it is apparent that the hyperinflation system 3 of the present invention, aside from continuously supplying air to the patient, can also facilitate simultaneously viewing of the pressure level inside the system 3 as indicated in the pressure gauge 321 and adjustment of the pressure through the pressure-releasing element 37 so as to maintain the system 3 at a suitable pressure level, thereby enhancing safe and convenient use of the present invention. Further, when suctioning of the phlegm of the patient is desired, there is no need to discontinue the supply of air to the patient, and the cover body 336 is simply removed from the third port 3312 to facilitate insertion of the suction tube through the third port 3312.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

We claim:

1. A hyperinflation system comprising:
an airbag having an air inlet and an air outlet;
a one-piece multi-way connector including a base, a first tube section projecting from a first side of said base and connected to said air outlet, a second tube section projecting from a second side of said base oppositely of said first tube section, and a pressure gauge projecting from said second side of said base adjacent to said second tube section; and
a multi-port adaptor including a first port connected fluidly to said second tube section, and a second port adapted for connection with a patient interface.

2. The hyperinflation system of claim 1, further comprising a pressure-releasing element connected to said second tube section for releasing air pressure inside said hyperinflation system.

3. The hyperinflation system of claim 2, wherein said second tube section defines an air passage, and has an arc-shaped aperture communicating fluidly with said air passage, said pressure-releasing element having a knob connected rotatably to said second tube section, and an arc-shaped vent hole provided in said knob and alignable with said arc-shaped aperture, said knob being rotatable to adjust the position of said arc-shaped vent hole relative to said arc-shaped aperture.

4. The hyperinflation system of claim 3, wherein said pressure-releasing element further has a pivot hole formed in said knob, said second tube section further having a pivot pin projecting outwardly from an outer face of said second tube section in proximity to said arc-shaped aperture and connected pivotally to said pivot hole.

5. The hyperinflation system of claim 2, further comprising an air supply tube which has one end extending through said airbag via said air inlet and said air outlet, said first and second tube sections, and into said first port, the other end of said air supply tube being adapted to be connected to an air supply source.

6. The hyperinflation system of claim 1, wherein said pressure gauge has a graduated pressure scale provided on an outer face thereof.

7. The hyperinflation system of claim 6, wherein said outer face of said pressure gauge is further provided with a column of pressure-indicating color levels proximate to said graduated pressure scale.

8. The hyperinflation system of claim 1, wherein said multi-port adaptor further includes a third port that opens in a direction opposite to said second port, and has a T-tube body including a fourth tube section having two opposite ends formed respectively with said second and third ports, a fifth tube section extending outwardly from said fourth tube section between said second and third ports and having said first port, and a first connecting tube inserted into said second tube section, said fifth tube section further having an extension connected rotatably to said first connecting tube, so that said fifth tube section is rotatable by an angle of 360° relative to said second tube section.

9. The hyperinflation system of claim 8, wherein said multi-port adaptor further includes a second connecting tube having one end connected rotatably to said fourth tube section in proximity to said second port and rotatable by an angle of 360° relative to said fourth tube section, the other end of said second connecting tube being adapted for connection with the patient interface.

10. The hyperinflation system of claim 8, wherein said third port is adapted for insertion of a suction tube, and is provided with a cover body to openably close said third port.
